(19) United States
(12) Patent Application Publication
(10) Pub. No.: US 2026/0069169 A1
(43) Pub. Date: Mar. 12, 2026

(54) Activity Recognition from Multiple Wearable Devices

(71) Applicant: Samsung Electronics Co., Ltd., Suwon-si (KR)

(72) Inventors: Nafiul Rashid, San Jose, CA (US); Xianghao Zhan, Palo Alto, CA (US); Ebrahim Nematihosseinabadi, San Jose, CA (US); Mohsin Ahmed, Sunnyvale, CA (US); Jilong Kuang, San Jose, CA (US)

(21) Appl. No.: 19/323,828

(22) Filed: Sep. 9, 2025

Related U.S. Application Data
(60) Provisional application No. 63/692,547, filed on Sep. 9, 2024.
(60) Provisional application No. 63/693,127, filed on Sep. 10, 2024.

(51) Int. Cl. A61B 5/11; A61B 5/00

(52) U.S. Cl. CPC A61B 5/1118; A61B 5/0024; A61B 5/1116; A61B 5/6817; A61B 5/6824; A61B 5/7207; A61B 5/7267; A61B 5/7275; A61B 2562/0219

(57) ABSTRACT

In one embodiment, a method includes accessing a current sensor signal from an inertial measurement unit (IMU) of an earbud in a current earbud orientation worn by a user and accessing a baseline-orientation IMU sensor signal for the earbud in a baseline earbud orientation. The method further includes estimating, for the earbud and based on the current sensor signal and the baseline-orientation IMU signal, an orientation transformation matrix that transforms the current sensor signal from the current earbud orientation to the baseline earbud orientation; and transforming the current sensor signal from the current earbud orientation to the baseline earbud orientation using the orientation transformation matrix.

### PRIORITY CLAIM

This application claims the benefit under 35 U.S.C. § 119 of U.S. Provisional Patent Application Nos. 63/692,547, filed Sep. 9, 2024, and 63/693,127, filed Sep. 10, 2024, each of which is incorporated by reference herein.

### TECHNICAL FIELD

This application generally relates to activity recognition from multiple wearable devices.

### BACKGROUND

Various sensors may be incorporated into consumer electronic devices, including devices that are worn on a user's body. For example, sensors may be included in devices such as a wrist-worn watch, a ring, an activity-tracker, a necklace, clothing, and/or head-worn devices such as headphones, earbuds, headbands, etc. Sensors embedded in such devices can include sensors that detect motion, position, EM waves, audio or other mechanical waves, and so on. For example, an inertial measurement unit may be embedded in a user-worn device, such as in a watch or in a set of earbuds.

### BRIEF DESCRIPTION OF THE DRAWINGS

FIG. 1 illustrates various activities and attempts to detect them using sensor fusion.

FIG. 2 illustrates an example architecture and corresponding techniques that improve activity predictions of the wearer using a pair of earbuds and a smartwatch.

FIG. 3 illustrates an example method for correcting earbud misalignment in a particular wearing session.

FIG. 4 illustrates an example modality-aware dynamic fusion process.

FIG. 5 illustrates an example of a stage-2 process of the implementation of FIG. 4.

FIG. 6 illustrates an example Stage-3 activity classification process of the implementation of FIG. 4.

FIG. 7 illustrates an example transition handler algorithm for confirming a predicted activity.

FIG. 8 illustrates an example computing system.

### DESCRIPTION OF EXAMPLE EMBODIMENTS

Fusing data from multiple sensors to detect user activities can improve activity recognition by levering the strengths of each sensor's detection capabilities while reducing the impact of each sensor's weaknesses. For example, a wrist-worn device such as a smartwatch is good for tracking activities that involve hand motions, such as walking, running and, jogging, but its prone to high noise due to hand motion, and tends to be placed far away from the center of body mass. Head-worn devices such as earbuds do not suffer from noise due to hand motions and do tend to move with the center of body mass, but they do not capture hand motions with the fidelity that wrist-worn devices do. Combining these sensor modalities to detect activity can improve the detection and categorization results.

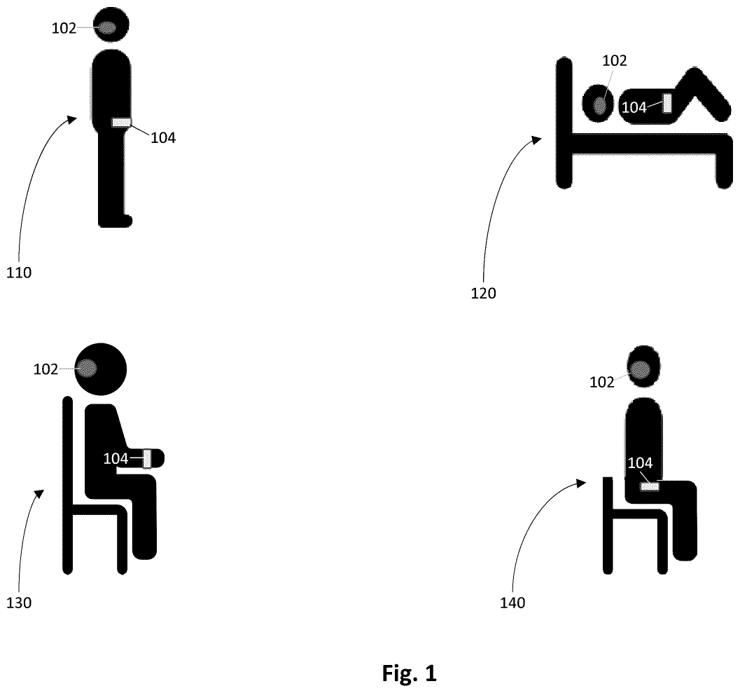

However, fusing wrist-worn and head-worn sensor output to detect a wearer's activity raises several challenges. For example, the orientation of a pair of earbuds can vary from wearer to wearer and even for a specific wearer across wearing sessions, affecting the performance of activity recognition due to earbud misalignment, and there is a scarcity of misalignment training data to train AI models to account for earbud orientation. As another example, FIG. 1 illustrates how certain activities can benefit from sensor fusion. For instance, if a person is wearing earbuds 102 and a smartwatch 104, then the earbuds have the same general orientation in poses 110, 130, and 140. In poses 110 and 140 the smartwatch has the same orientation, which is different than the orientation of the smartwatch in poses 130 and 140. Therefore, by fusing sensor data, various user poses can often be determined. However, a user is sitting in both poses 130 and 140, yet smartwatch 104 records a different orientation in each pose, which can create challenges for accurately identifying the corresponding activity (which is the same in both poses). Likewise, multi-stage dynamic fusion may include inefficient (e.g., redundant) processes.

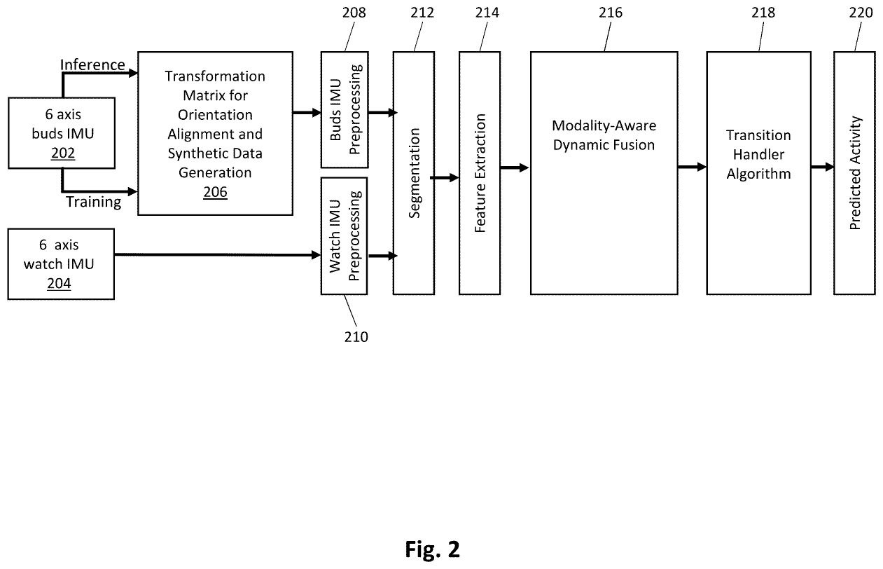

FIG. 2 illustrates an example architecture and corresponding techniques that improve activity predictions for the wearer using a pair of earbuds and a smartwatch. In the example of FIG. 2, an IMU 202 of an earbud (e.g., a 6-axis IMU containing an accelerometer and a gyroscope, although other embodiments may use only one such sensor) collects sensor signals in the earbud's current orientation. Likewise, an IMU 204 of a smartwatch collects sensor signals. The sensor signals from the earbud IMU 202 are adjusted using a transformation matrix 206 during inference to correct earbud misalignment. And as explained below, transformation matrix 206 may also or alternatively be used to generate training IMU 202 data for machine-learning model training during the training phase.

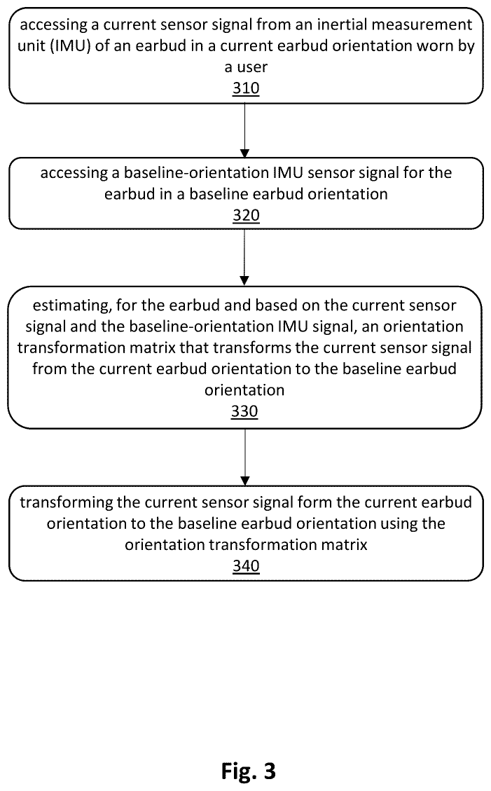

FIG. 3 illustrates an example method for correcting earbud misalignment during a particular wearing session. Step 310 of the example method of FIG. 3 includes accessing a current sensor signal from an inertial measurement unit (IMU) of an earbud in a current earbud orientation and worn by a user. For instance, when a user first inserts an earbud into their ear, then step 310 may be performed to access current IMU data from the earbud in that current earbud orientation. Step 320 of the example method of FIG. 3 includes accessing a baseline-orientation IMU sensor signal for the earbud having a baseline earbud orientation. This baseline-orientation IMU sensor signal may come from a calibration process; for example, the user may perform a single calibration process for the earbud that determines the baseline orientation, which in particular embodiments is the earbud orientation that aligns earbuds to the wearer's anatomical coordinates. As a result, in the preceding example the baseline orientation is the earbud orientation in which the earbud is aligned with the anatomical coordinate system; however, during regular use a user will much more frequently place the earbuds at least somewhat misaligned to this baseline orientation.

Step 330 of the example method of FIG. 3 includes estimating, for the earbud and based on the current sensor signal and the baseline-orientation IMU signal, an orientation transformation matrix that transforms the current sensor signal from the current earbud orientation to the baseline earbud orientation. For instance, the orientation transformation matrix R may be defined by three Euler angels yaw (α), pitch (β), roll (γ) according to:

$$R = {R}_{z} ( α ) {R}_{y} ( β ) {r}_{x} ( γ ) = [ \begin{matrix}cos α & - sin α & 0 \\ sin α & cos α & 0 \\ 0 & 0 & 1\end{matrix} ] [ \begin{matrix}cos β & 0 & sin β \\ 0 & 1 & 0 \\ - sin β & 0 & cos β\end{matrix} ] [ \begin{matrix}1 & 0 & 0 \\ 0 & cos γ & - sin γ \\ 0 & sin γ & cos γ\end{matrix} ] = [ \begin{matrix}cos α cos β & cos α sin β sin γ - sin α cos γ & cos α sin β cos γ + sin α sin γ \\ sin α sin β & sin α sin β sin γ + cos α cos γ & sin α sin β cos γ - cos α sin γ \\ - sin β & cos β sin γ & cos β cos γ\end{matrix} ]$$

To align an arbitrary current earbud orientation to the baseline earbud orientation, particular embodiments of step 330 include estimating the Euler angles that minimize the mean squared error (MSE) between the transformed linear acceleration of the current sensor signal and the baseline linear acceleration from the baseline orientation signal.

Particular embodiments may estimate the orientation transformation matrix using a grid search method. For example, a first, thorough grid-search approach may perform a grid search on each of the three Euler angles (three degrees of freedom) with a grid size of, e.g., 5 from −180° to 180°. Other embodiments may use a larger or smaller grid size, which reduces the computational complexity of a very fine grid search (e.g., a thorough grid search with a grid size of 1° for three Euler angles results in 47,045,881 combinations). Other embodiments may use a limited, more efficient (but possibly less accurate) grid search approach that performs a grid search on only one or two of the three possible Euler angles, while the Euler angles for which a grid search is not performed are set to 0°.

Particular embodiments may estimate the orientation transformation matrix using an iterative Markov Chain Monte Carlo (MCMC) sampling technique. For example, starting from an initial point in the three-dimensional Euler angle space, the MCMC techniques makes a random movement with a step size δ. A movement towards the optimization goal (e.g., lowest mean squared error) is always accepted; otherwise, the movement is accepted with a small probability p. The movement continues until one stopping criteria is reached. In particular embodiments, the stopping criteria may include (1) a maximum number of steps T; (2) meeting a MSE threshold requirement MSEthreshold; or (3) the MSE changes by less than a minimum MSE change in a given step tol. In particular embodiments, the sampling step size δ is used as an MCMC hyperparameter. The remaining hyperparameter may be set as, for example: p=0.1; T=10,000; tol=1e−7, and MSEthreshold in particular embodiments is set to 0.0384 meters²/seconds⁴ (i.e., set to an RMSE of about ≤2% g), which accounts for human breathing and other inevitable body motion.

Step 340 of the example method of FIG. 3 includes transforming the current sensor signal from the current earbud orientation to the baseline earbud orientation using the orientation transformation matrix. For instance, the orientation matrix R described above may be used to transform the current senor signal in its current earbud orientation to what the signal is in the baseline orientation frame of reference, which as described above, is often aligned with the head-to-foot axis of the wearer.

In particular embodiments, a technique used to estimate the orientation transformation matrix can be used to generate synthetic data for various earbuds orientations. However, unlike orientation alignment, here the Euler angles are estimated to maximize the mean squared error (MSE) between the transformed linear acceleration and baseline linear acceleration to have synthetic data at various orientations. Using linear acceleration signals collected from different subjects and with different degrees of earbuds rotation relative to the baseline (e.g., 30, 60, 90, 120, . . . , 360 degrees counterclockwise rotation with respect to the baseline earbuds orientation), the coordination transformation matrices can be estimated with MCMC or grid search. Then, the transformation matrices can be applied to augment the IMU signals in datasets strictly collected under standard earbuds orientation to generate synthetic rotated earbuds IMU data. This augmentation can then be used to train machine-learning models to perform orientation correction in the presence of earbuds orientation heterogeneity. Since the synthetic dataset generation is not as time sensitive as the orientation alignment, particular embodiments may use a grid search technique for most accurate dataset generation. However, MCMC can also be used for its efficiency over grid search.

After collecting sensor data and performing earbud alignment, then smartwatch IMU signal preprocessing 210 and earbud IMU preprocessing 208 occurs. For instance, the sampling period for a smartwatch may be set to 20 ms (50 Hz), while for a pair of earbuds the sampling period may vary between 18 to 22 ms. Particular embodiments therefore package every 20 samples for both watch and earbuds and transmit the package to a connected device, e.g., as a burst to a connected smartphone through Bluetooth. Since the sampling rate for the earbuds is not exactly 50 Hz, the first step is to resample data from both devices to a constant rate (i.e., to 50 Hz in this example).

After preprocessing, step 212 includes segmenting the packaged data. The collected data from both earbuds and the smartwatch are segmented with, for example, a window size of 5 seconds of data with a step size of 1 second, although other window and steps sizes may be used. As the data is collected at 50 Hz (in this example) from both buds and watch, each window segment consists of 250 data samples and there is an overlap of 200 samples between the consecutive windows.

Step 214 includes feature extraction from the segmented data. For each sample of the segmented window, particular embodiments calculate the magnitude of the accelerometer and gyroscope values from their corresponding X, Y, and Z axis for both watch and earbuds. The calculated magnitude along with the X, Y, and Z axis of the accelerometer and gyroscope for both the watch and buds makes a total of 16 input channels to analyze (3 channels from the earbud accelerometer, 3 from the earbud gyroscope, 3 from the watch accelerometer, 3 from the watch gyroscope, and 1 magnitude channel from each of those three-channel groups). Particular embodiments extract 11 statistical features from each of those 16 input channels that result in 176 features (here, 88 from the smartwatch and 88 from the earbuds). For instance, in particular embodiments extracted features can include mean, absolute mean, minimum, maximum, absolute maximum, variance, crest, kurtosis, skewness, range, and interquartile range. Additionally, particular embodiments calculate 81 earbuds-smartwatch coordination features which includes the angle between the smartwatch and earbuds (this is one of the 81 features), and 4 correlation features (Pearson correlation, maximum value of cross-correlation, lag of cross-correlation, and mean value of coherence) from each of 20 channels that are made up of 10 pairs of correlation signal channels (9 pairs each of non-magnitude channels from the earbuds and the smartwatch, and 1 pair of magnitude channels from earbuds and smartwatch) for each of the 2 IMU sensors (accelerometer and gyroscope). In other words, each x, y, and z accelerometer signal from the earbud accelerometer is paired with each of the x, y, and z accelerometer signal from the smartwatch, resulting in the 9 pairs of non-magnitude channels for that accelerometer.

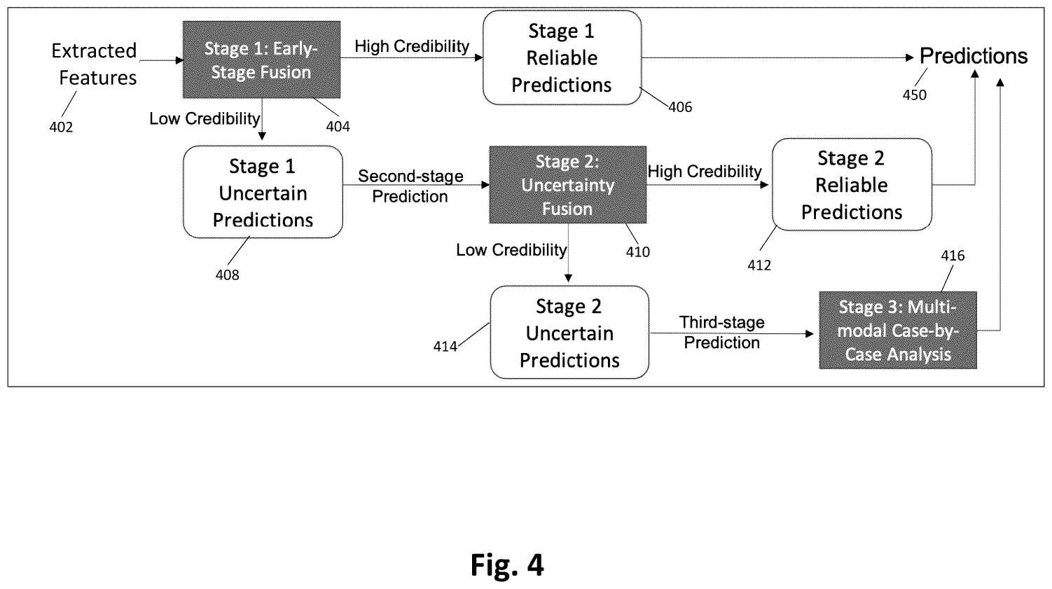

After feature extraction, the example technique of FIG. 2 includes a modality-aware dynamic fusion process 216. FIG. 4 illustrates an example modality-aware dynamic fusion process that includes (1) an early-stage fusion decision block that outputs an activity prediction if the prediction credibility is above a threshold; (2) a second-stage uncertain fusion decision block that uses separate classification models for each sensor modality and (3) a third-stage decision block that takes into account each modality's accuracy in predicting activities. The example embodiment of FIG. 4 uses conformal prediction arrive a prediction credibility for predicted activities.

Conformal prediction (CP) is a quantification of prediction uncertainty. As a brief introduction to conformal prediction, assuming that there are N training data samples in D-dimensional feature space (xᵢ∈ [symbol] , i=1, 2, . . . , N), T test data samples (xⱼ∈ [symbol] , j=1, 2, . . . , T) from K classes (yᵢ∈{1, 2, . . . , K}), conformal prediction first trains a model f to calculate the nonconformity measurement \vec{α}ₗ∈ [symbol] , i=1, 2, . . . , N based on the training dataset (xᵢ, yᵢ), i=1, 2, . . . , N, which reflects the model's opinion on the likelihood of the feature-label combination (xᵢ, yᵢ) (Eqn. 1). Here, the nonconformity measurement \vec{α}ₗ is a vector that includes the nonconformity measurement for every possible label, with each element

$${α}_{i}^{{y}_{i}}$$

representing the nonconformity measurement when a particular label (yᵢ) is paired with the feature (xᵢ).

$$f : ( {x}_{i} , {f}_{i} ) \to {α}_{i}^{{y}_{i}} ∈ ℝ \qquad ( 1 )$$

The nonconformity measurement can be calculated based on predicted probability P(Y=k|X), k=1, 2, . . . , K of a typical classification algorithm like logistic regression (CP-LR), linear discriminant analysis (CP-LDA), random forest (CP-RF) and artificial neural network (CP-ANN), which makes the conformal prediction easy to implement as most classification algorithms can model the conditional probability P(Y=k|X), k=1, 2, . . . , K. The nonconformity measurement can also be calculated in a heuristic manner such as using the ratio between the sum of distances among m homogeneous samples and the sum of distances among m heterogeneous samples on the Euclidean distance in K-nearest neighbors (CP-KNN). Then, the nonconformity measurement values of the training data

$${α}_{i}^{{y}_{i}}$$

(feature xᵢ and ground-truth label yᵢ) are recorded for the follow-up calibration process.

The example of FIG. 4 uses a nonconformity measurement calculation method based on the classifier's predicted probability (Eqn. 2), although other nonconformity techniques may be used. For a given sample with feature x, the nonconformity measurement for label y=k, k=1, 2, . . . , K, α^{y=k} is computed as:

$${α}^{y = k} = 0.5 - \frac{P ( Y = k \vert x ) - max ( {P}_{Y != k} ( Y \vert x ) )}{2} \qquad ( 2 )$$

During inference, every possible label (y=k, k=1, 2, . . . , K) is paired with j-th test feature

$${x}_{j}^{*}$$

=1, 2, . . . , T, and the P-values are calculated by calibrating a nonconformity measurement of a particular test feature-label with regard to the reference distribution of the training nonconformity measurement values according to Eqn. 3. The P-values associated with a particular label quantifies how well a particular feature-label combination conforms to the reference ground-truth distribution of the training dataset. The P-value reflects the likelihood of observing a true feature-label combination with a nonconformity measurement value larger than the test nonconformity measurement value (in another word, “stranger” than the test observation from the perspective of the training data), under the distribution of the training data nonconformity measurement values.

$${p}_{j}^{y} = \frac{\vert \{ m = 1 , , N \} {α}_{m}^{y} \geq {α}_{j}^{y} \vert + 1}{N + 1} \qquad ( 3 )$$

The final prediction output by a conformal predictor is the label with the highest P-value. Based on the conformal prediction, the prediction credibility is defined as the highest P-value among all labels credibility=max(p^{y}), y=1, 2, . . . , K, which is usually used as the prediction uncertainty metric in the previous literature. In the example of FIG. 4, the credibility quantifies the reliability for the predicted label by investigating how well the predicted label conforms to the training data distribution. This prediction credibility is used to implement the modality-aware dynamic fusion method of FIG. 4 that includes three stages: early-stage (first stage), uncertainty late-stage (second stage), and multi-modal case-by-case analysis (third stage).

In the example of FIG. 4, extracted features 402 from the feature extraction process are passed to stage-1 fusion process 404. At this first stage predictor, one trained classification model combines the features from the different sensor modalities (e.g., 257 features: 88 from earbuds, 88 from smartwatches and 81 from bud-watch coordinates) and classifies the probability of different activities from the features. In the example of FIG. 4, the probabilities are prediction credibility values quantified using conformal prediction. If any predicted activity class has a credibility of more than a threshold value, e.g., more than a threshold value of 0.5, then the class with the highest such credibility is regarded as a reliable, high-credibility activity prediction 406 and is output as the prediction 450 of the modality-aware dynamic fusion process. If no such activity classification has a credibility that is greater than the threshold value, then modality-aware dynamic fusion process moves to stage 2.

In the example of FIG. 4, the stage 1 predictions 408 (none of which are sufficiently credible to be used as a stage-1 prediction) are passed to the second stage process 410. This second stage uses a separate classification model trained specifically for each sensor modality and, in particular embodiments, each combination of sensor modalities. Prediction p-values are calculated for each classification model and then averaged for the final prediction and final credibility. If a prediction has a credibility higher than a threshold value, e.g., higher than 0.5, then the highest such prediction is output as the final predicted activity. If not, then the process enters stage 3, described below.

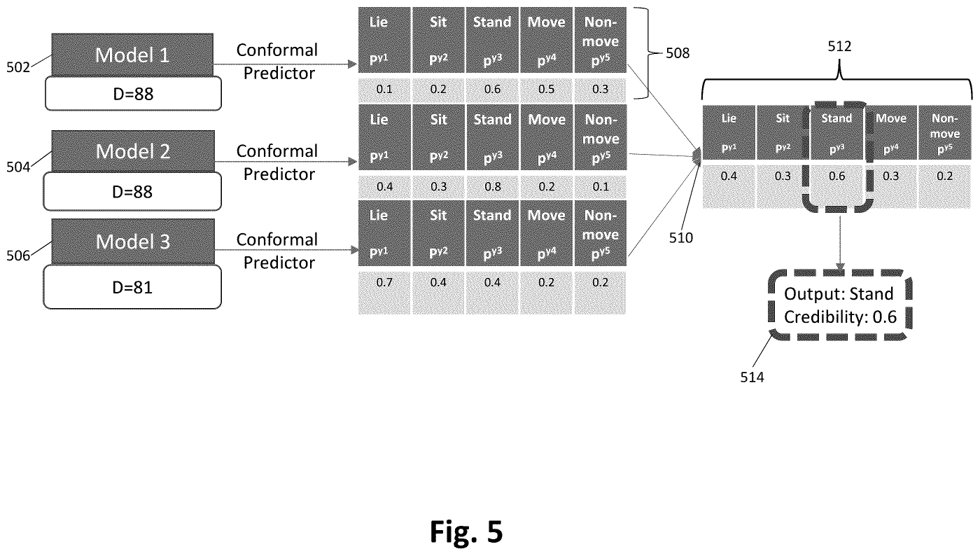

FIG. 5 illustrates an example of a stage-2 process of the implementation of FIG. 4. In the example of FIG. 5, classification model 502 is used to classify features (88 features, in this example) from the earbud IMU. Classification model 502 outputs a predicted p-value for each class, as shown in output 508 for classification model 502. For instance, here the activity labels include “lie” “sit” “stand” “move” and “non-move,” although other activity classification labels may be used as appropriate, in conjunction with the training labels used to train the classification model. As shown in output 508, along with each classification label is an output p-value for that activity given the input data.

In the example of FIG. 5, classification model 504 is used to output p-values for the activity classes based on features extracted from the smartwatch, while classification model 506 outputs p-values for the activity labels based on features from the combination earbud/smartwatch features. In the example of FIG. 5, the output p values from each classification model are then averaged 510 for each activity label, resulting in stage-2 predictions 512. Then, if a stage-2 prediction is greater than a threshold credibility value (e.g., greater than 0.5), then the highest such prediction is output as the final prediction 514 for the activity classification (i.e., is a reliable stage-2 prediction 412 of FIG. 4), and this prediction becomes the final prediction 450 for the activity in the example of FIG. 4. If no such p-value exists at the end of stage 2 of FIG. 4 such that only unreliable predictions 414 exist, then the process moves to stage 3.

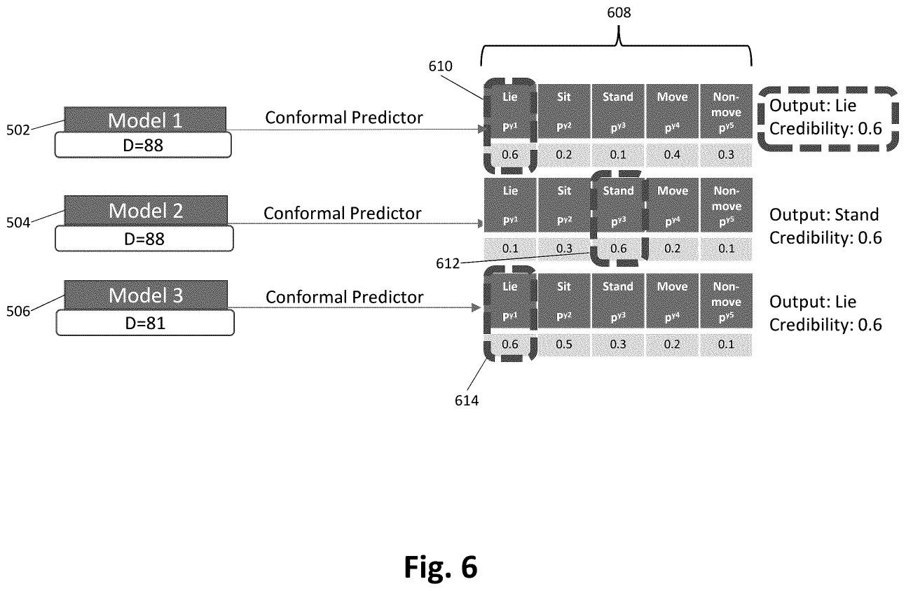

FIG. 6 illustrates an example Stage-3 activity classification process. In the example of FIG. 6, the classification models discussed in FIG. 5 provide prediction output 608 from the features, as described in connection with FIG. 5. In practice, prediction output 508 and output 608 would be the same for a given inference task, but for illustration purposes these outputs differ in FIGS. 5 and 6.

In stage 3, each classification model is associated with particular class labels for which that classification model provides the best prediction accuracy. For example, this association may be made based on the per-class F1 score on a validation set. For example, IMU data from an earbud is most reliable at predicting “lie” and “move” activities, while IMU data from a smartwatch is most reliable at predicting “sit” and “non-move” activities, and the combination is most reliable at predicting “stand” activities. Then, the most credible output from each classification model is identified; i.e., in the example of FIG. 6, output 610 has the highest p value in the output from classification model 502, output 612 has the highest p value in the output from classification model 504, and output 614 has the highest p value in the output from classification model 506. However, output 612 corresponds to a class (stand) that is not what classification model 504 is most accurate at, and likewise output 614 corresponds to a class (lie) that is not what classification model 506 is most accurate at. In contrast, output 610 does correspond to a class (lie) that classification model 610 is most accurate at. Therefore, even though each classification model has predicted a class with the same p value, the classification from model 502 is selected as the stage-3 prediction because the most credible class is one that corresponds what model 502 is most accurate at predicting.

In the example of FIG. 2, after a final prediction is provided from a modality-aware dynamic fusion process 216, then a transition handler algorithm 218 is used to finalize the activity prediction. This transition handler algorithm can be useful in ensuring that changes in activity states are reliable given the detected motion from the various sensor modalities. For example, it is very difficult to move from a sitting position to a standing position without moving both the head and the hands, and therefore an activity prediction of “stand” that follows a previous prediction of “sit” should entail movement from both the head and the hands to make that predicted activity transition credible.

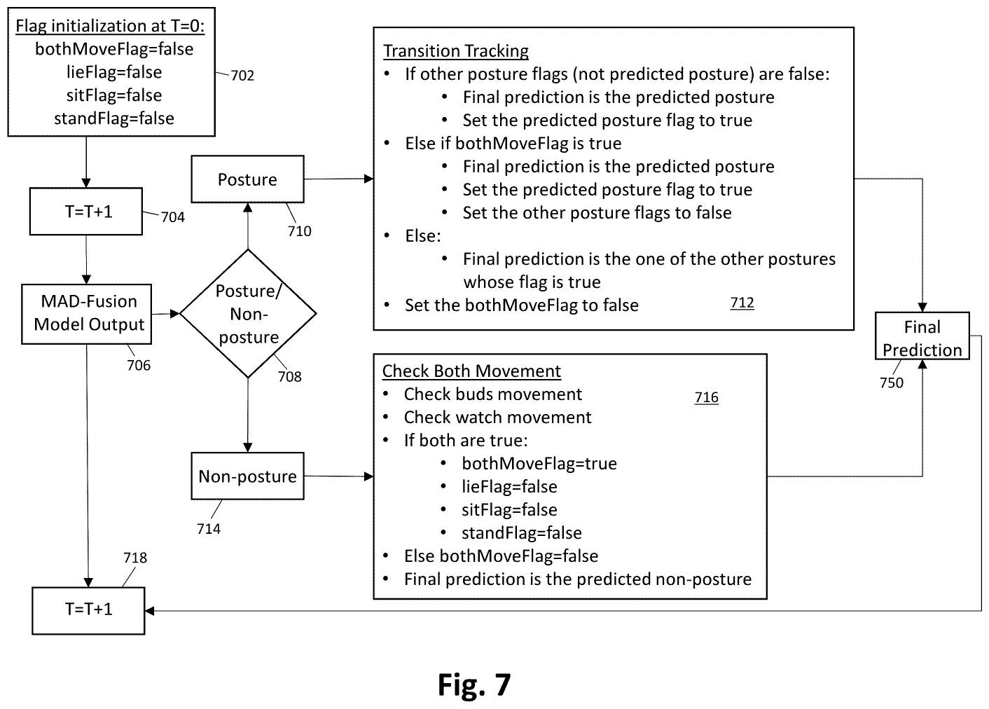

FIG. 7 illustrates an example transition handler algorithm. First, flag initialization step 702 at iteration T=0 sets the corresponding flag for each posture activity (in this example, Lie, Stand, Sit, although more or fewer activity labels may be used) to False. Additionally, a ‘bothMoveFlag,’ which tracks the movements of both devices to help understand the transition from one posture to another despite random movements in the devices is also set to False. Iteration T is then incremented by one in step 704, and the output from the modality-aware dynamic fusion process 706 is accessed. Decision block 708 determines whether the predicted activity from the modality-aware dynamic fusion process is a posture-type activity or a non-posture type activity (e.g., move or non-move, in this example). If the predicted activity corresponds to a posture, then transition tracking block 712 is used.

In the example of FIG. 7, transition tracking block 712 first involves checking the flag values at previous iteration T−1 to determine whether the posture-class flags for postures other than predicted posture are false. If they are false, this implies that the previous activity at T−1 was not from a different posture class. In that case, the current predicted posture at iteration T is considered as the final predicted class and its corresponding flag is set to True. Otherwise, if a posture-class flag for a posture other than the predicted one at iteration T−1 is not false, then transition tracking block 712 checks whether the ‘bothMoveFlag’ at T−1 is true. If this flag is True, then this implies that the previous activity involved body movements. Then, the final prediction is the predicted posture class, all other posture flags are set to False, and the bothMoveFlag is also set to False. Finally, if another posture flag is not False and the bothMoveFlag is not True, then the previous activity should be a non-posture activity resulting from random movements in one of the devices. Therefore, the current predicted activity should be one of the postures who flag was True at the last prediction iteration T−1. Thus, the final output is one of these postures. As a result, the transition tracking considers the current prediction from the MAD-Fusion process as the final prediction only if this prediction corresponds to movement in both devices, validating the transition from one activity to another and avoiding false classification due to random movements.

If the predicted activity from the modality-aware dynamic fusion process 706 corresponds to a non-posture class 714, then the transition handler algorithm moves to movement analysis block 716. In the example of FIG. 7, activity analysis block 716 first checks if there is movement detected by each of the earbud and the smartwatch. This is done by checking the output of the classification models for the earbuds and the smartwatch separately (as shown in the second stage of MAD-Fusion method). If both of the models output the current activity as ‘move’ then block 716 sets the ‘bothMoveFlag’ to True and sets all posture flags to False. Otherwise, the ‘bothMoveFlag’ is set False. This ‘bothMoveFlag’ is used by block 712, described above. The final prediction 750 from block 716 is the predicted non-posture activity from step 706. After a final prediction 750 is made (which corresponds to final prediction step 220 in the example of FIG. 2), then the step counter T is incremented in step 718, and the process returns to step 706 for the next predicted activity.

In particular embodiments, some steps of the example implementation of FIG. 2 and described herein may be performed by a wearable device or may be performed by a connected device (e.g., a client device such as a smartphone or a personal computer, or by a server device). For example, the earbud may acquire IMU signals in step 202 and a smartwatch may acquire its signals in step 204. Each of steps 206-220 may be performed by any of either wearable device, a client device (e.g., a mobile device such as a smartphone), or a server device. For instance, a smartphone may access the acquired IMU signals from each wearable device and then perform the transformation, processing, fusion, and prediction processes of steps 206-220. Other embodiments may use a server device to perform some or all of those steps, and the server device may communicate with a client device or directly with a wearable device, in particular embodiments. In particular embodiments, classification models such those discussed with respect to FIGS. 5 and 6 may be deployed on a wearable device, a client device, or a server device, although a wearable device's typically more limited resources usually means the such classification models would typically be deployed on a client device or on a server device.

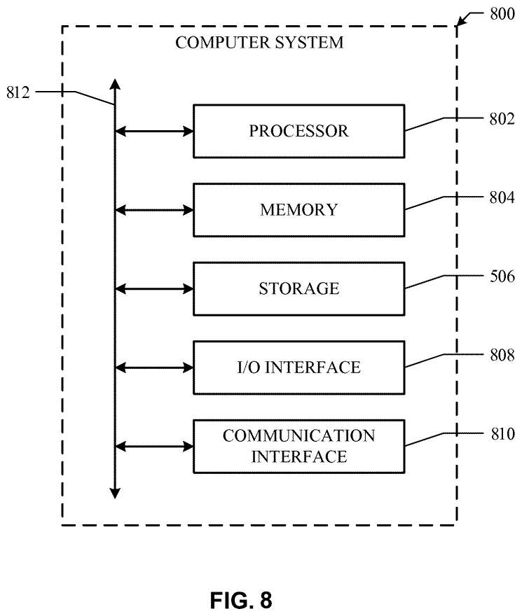

FIG. 8 illustrates an example computer system 800. In particular embodiments, one or more computer systems 800 perform one or more steps of one or more methods described or illustrated herein. In particular embodiments, one or more computer systems 800 provide functionality described or illustrated herein. In particular embodiments, software running on one or more computer systems 800 performs one or more steps of one or more methods described or illustrated herein or provides functionality described or illustrated herein. Particular embodiments include one or more portions of one or more computer systems 800. Herein, reference to a computer system may encompass a computing device, and vice versa, where appropriate. Moreover, reference to a computer system may encompass one or more computer systems, where appropriate.

This disclosure contemplates any suitable number of computer systems 800. This disclosure contemplates computer system 800 taking any suitable physical form. As example and not by way of limitation, computer system 800 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, or a combination of two or more of these. Where appropriate, computer system 800 may include one or more computer systems 800; be unitary or distributed; span multiple locations; span multiple machines; span multiple data centers; or reside in a cloud, which may include one or more cloud components in one or more networks. Where appropriate, one or more computer systems 800 may perform without substantial spatial or temporal limitation one or more steps of one or more methods described or illustrated herein. As an example and not by way of limitation, one or more computer systems 800 may perform in real time or in batch mode one or more steps of one or more methods described or illustrated herein. One or more computer systems 800 may perform at different times or at different locations one or more steps of one or more methods described or illustrated herein, where appropriate.

In particular embodiments, computer system 800 includes a processor 802, memory 804, storage 806, an input/output (I/O) interface 808, a communication interface 810, and a bus 812. Although this disclosure describes and illustrates a particular computer system having a particular number of particular components in a particular arrangement, this disclosure contemplates any suitable computer system having any suitable number of any suitable components in any suitable arrangement.

In particular embodiments, processor 802 includes hardware for executing instructions, such as those making up a computer program. As an example and not by way of limitation, to execute instructions, processor 802 may retrieve (or fetch) the instructions from an internal register, an internal cache, memory 804, or storage 806; decode and execute them; and then write one or more results to an internal register, an internal cache, memory 804, or storage 806. In particular embodiments, processor 802 may include one or more internal caches for data, instructions, or addresses. This disclosure contemplates processor 802 including any suitable number of any suitable internal caches, where appropriate. As an example and not by way of limitation, processor 802 may include one or more instruction caches, one or more data caches, and one or more translation lookaside buffers (TLBs). Instructions in the instruction caches may be copies of instructions in memory 804 or storage 806, and the instruction caches may speed up retrieval of those instructions by processor 802. Data in the data caches may be copies of data in memory 804 or storage 806 for instructions executing at processor 802 to operate on; the results of previous instructions executed at processor 802 for access by subsequent instructions executing at processor 802 or for writing to memory 804 or storage 806; or other suitable data. The data caches may speed up read or write operations by processor 802. The TLBs may speed up virtual-address translation for processor 802. In particular embodiments, processor 802 may include one or more internal registers for data, instructions, or addresses. This disclosure contemplates processor 802 including any suitable number of any suitable internal registers, where appropriate. Where appropriate, processor 802 may include one or more arithmetic logic units (ALUs); be a multi-core processor; or include one or more processors 802. Although this disclosure describes and illustrates a particular processor, this disclosure contemplates any suitable processor.

In particular embodiments, memory 804 includes main memory for storing instructions for processor 802 to execute or data for processor 802 to operate on. As an example and not by way of limitation, computer system 800 may load instructions from storage 806 or another source (such as, for example, another computer system 800) to memory 804. Processor 802 may then load the instructions from memory 804 to an internal register or internal cache. To execute the instructions, processor 802 may retrieve the instructions from the internal register or internal cache and decode them. During or after execution of the instructions, processor 802 may write one or more results (which may be intermediate or final results) to the internal register or internal cache. Processor 802 may then write one or more of those results to memory 804. In particular embodiments, processor 802 executes only instructions in one or more internal registers or internal caches or in memory 804 (as opposed to storage 806 or elsewhere) and operates only on data in one or more internal registers or internal caches or in memory 804 (as opposed to storage 806 or elsewhere). One or more memory buses (which may each include an address bus and a data bus) may couple processor 802 to memory 804. Bus 812 may include one or more memory buses, as described below. In particular embodiments, one or more memory management units (MMUs) reside between processor 802 and memory 804 and facilitate accesses to memory 804 requested by processor 802. In particular embodiments, memory 804 includes random access memory (RAM). This RAM may be volatile memory, where appropriate Where appropriate, this RAM may be dynamic RAM (DRAM) or static RAM (SRAM). Moreover, where appropriate, this RAM may be single-ported or multi-ported RAM. This disclosure contemplates any suitable RAM. Memory 804 may include one or more memories 804, where appropriate. Although this disclosure describes and illustrates particular memory, this disclosure contemplates any suitable memory.

In particular embodiments, storage 806 includes mass storage for data or instructions. As an example and not by way of limitation, storage 806 may include a hard disk drive (HDD), a floppy disk drive, flash memory, an optical disc, a magneto-optical disc, magnetic tape, or a Universal Serial Bus (USB) drive or a combination of two or more of these. Storage 806 may include removable or non-removable (or fixed) media, where appropriate. Storage 806 may be internal or external to computer system 800, where appropriate. In particular embodiments, storage 806 is non-volatile, solid-state memory. In particular embodiments, storage 806 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 806 taking any suitable physical form. Storage 806 may include one or more storage control units facilitating communication between processor 802 and storage 806, where appropriate. Where appropriate, storage 806 may include one or more storages 806. Although this disclosure describes and illustrates particular storage, this disclosure contemplates any suitable storage.

In particular embodiments, I/O interface 808 includes hardware, software, or both, providing one or more interfaces for communication between computer system 800 and one or more I/O devices. Computer system 800 may include one or more of these I/O devices, where appropriate. One or more of these I/O devices may enable communication between a person and computer system 800. As an example and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 808 for them. Where appropriate, I/O interface 808 may include one or more device or software drivers enabling processor 802 to drive one or more of these I/O devices. I/O interface 808 may include one or more I/O interfaces 808, where appropriate. Although this disclosure describes and illustrates a particular I/O interface, this disclosure contemplates any suitable I/O interface.

In particular embodiments, communication interface 810 includes hardware, software, or both providing one or more interfaces for communication (such as, for example, packet-based communication) between computer system 800 and one or more other computer systems 800 or one or more networks. As an example and not by way of limitation, communication interface 810 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 810 for it. As an example and not by way of limitation, computer system 800 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 800 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computer system 800 may include any suitable communication interface 810 for any of these networks, where appropriate. Communication interface 810 may include one or more communication interfaces 810, where appropriate. Although this disclosure describes and illustrates a particular communication interface, this disclosure contemplates any suitable communication interface.

In particular embodiments, bus 812 includes hardware, software, or both coupling components of computer system 800 to each other. As an example and not by way of limitation, bus 812 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 812 may include one or more buses 812, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

This disclosure contemplates a system that includes one or more non-transitory computer readable storage media storing instructions; and one or more processors coupled to the one or more non-transitory computer readable storage media and operable to execute the instructions to perform certain functions includes embodiments in which those functions are performed by a single processor, embodiments in which those functions are performed by multiple processors that each perform all the functions, and embodiments in which those functions are performed by multiple processors (e.g., in separate computing devices) where each processor performs at least one function but less than all recited functions.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend.

What is claimed is:

1. A method comprising:
accessing a current sensor signal from an inertial measurement unit (IMU) of an earbud in a current earbud orientation worn by a user;
accessing a baseline-orientation IMU sensor signal for the earbud in a baseline earbud orientation;
estimating, for the earbud and based on the current sensor signal and the baseline-orientation IMU signal, an orientation transformation matrix that transforms the current sensor signal from the current earbud orientation to the baseline earbud orientation; and
transforming the current sensor signal from the current earbud orientation to the baseline earbud orientation using the orientation transformation matrix.

2. The method of claim 1, wherein estimating the orientation transformation matrix comprises estimating the orientation transformation matrix based on a grid search.

3. The method of claim 1, wherein estimating the orientation transformation matrix comprises estimating the orientation transformation matrix based on an iterative Markov chain Monte Carlo sampling until a stopping condition occurs.

4. The method of claim 3, wherein the stopping condition comprises an error threshold that corresponds to motion due to human breathing.

5. The method of claim 1, further comprising:
extracting a plurality of features from (1) the transformed current sensor signal from the IMU of the earbud and from (2) an IMU sensor signal of a wrist-worn device; and
predicting an activity of the user based on the extracted features.

6. The method of claim 5, further comprising:
determining, by a trained activity classification model and based on the extracted features, an activity prediction credibility for each a plurality of activity classes, wherein each credibility is based on a conformal prediction; and
determining whether any activity prediction credibility exceeds a threshold credibility value; and
when at least one activity prediction credibility exceeds the threshold credibility value, then selecting the activity class corresponding to the highest such activity prediction credibility as the predicted activity of the user.

7. The method of claim 6, further comprising:
when no activity prediction credibility exceeds the threshold credibility value, then determining, by each of a plurality of modality-specific trained activity classification models, an activity prediction credibility for each of the plurality of activity classes, wherein each of the plurality of modality-specific trained activity classification models corresponds to one of the earbud, the wrist-worn device, or a combination of the earbud and the wrist-worn device;
averaging, for each of the plurality of activity classes, the activity prediction credibility from each of the plurality of modality-specific trained activity classification models;
determining whether any averaged activity prediction credibility exceeds the threshold credibility value; and
when at least one averaged activity prediction credibility exceeds the threshold credibility value, then selecting the activity class corresponding to the highest such averaged activity prediction credibility as the predicted activity of the user.

8. The method of claim 5, wherein predicting the activity of the user comprises determining, by an activity transition handler, the predicted activity of the user.

9. A system comprising:
one or more non-transitory computer readable storage media storing instructions, and one or more processors coupled to the one or more non-transitory computer readable storage media and operable to execute the instructions to:
access a current sensor signal from an inertial measurement unit (IMU) of an earbud in a current earbud orientation worn by a user;
access a baseline-orientation IMU sensor signal for the earbud in a baseline earbud orientation;
estimate, for the earbud and based on the current sensor signal and the baseline-orientation IMU signal, an orientation transformation matrix that transforms the current sensor signal from the current earbud orientation to the baseline earbud orientation; and
transform the current sensor signal from the current earbud orientation to the baseline earbud orientation using the orientation transformation matrix.

10. The system of claim 9, wherein estimating the orientation transformation matrix comprises estimating the orientation transformation matrix based on a grid search.

11. The system of claim 9, wherein estimating the orientation transformation matrix comprises estimating the orientation transformation matrix based on an iterative Markov chain Monte Carlo sampling until a stopping condition occurs.

12. The system of claim 11, wherein the stopping condition comprises an error threshold that corresponds to motion due to human breathing.

13. The system of claim 9, further comprising one or more processors that are operable to execute the instructions to:
extract a plurality of features from (1) the transformed current sensor signal from the IMU of the earbud and from (2) an IMU sensor signal of a wrist-worn device; and
predict an activity of the user based on the extracted features.

14. The system of claim 13, further comprising one or more processors that are operable to execute the instructions to:
determine, by a trained activity classification model and based on the extracted features, an activity prediction credibility for each a plurality of activity classes, wherein each credibility is based on a conformal prediction; and
determine whether any activity prediction credibility exceeds a threshold credibility value; and
when at least one activity prediction credibility exceeds the threshold credibility value, then select the activity class corresponding to the highest such activity prediction credibility as the predicted activity of the user.

15. The system of claim 14, further comprising one or more processors that are operable to execute the instructions to:
when no activity prediction credibility exceeds the threshold credibility value, then determine, by each of a plurality of modality-specific trained activity classification models, an activity prediction credibility for each of the plurality of activity classes, wherein each of the plurality of modality-specific trained activity classification models corresponds to one of the earbud, the wrist-worn device, or a combination of the earbud and the wrist-worn device;
average, for each of the plurality of activity classes, the activity prediction credibility from each of the plurality of modality-specific trained activity classification models;
determine whether any averaged activity prediction credibility exceeds the threshold credibility value; and
when at least one averaged activity prediction credibility exceeds the threshold credibility value, then select the activity class corresponding to the highest such averaged activity prediction credibility as the predicted activity of the user.

16. The system of claim 13, wherein predicting the activity of the user comprises determining, by an activity transition handler, the predicted activity of the user.

17. One or more non-transitory computer-readable storage media comprising instructions that are operable when executed by one or more processors to:
access a current sensor signal from an inertial measurement unit (IMU) of an earbud in a current earbud orientation worn by a user;
access a baseline-orientation IMU sensor signal for the earbud in a baseline earbud orientation;
estimate, for the earbud and based on the current sensor signal and the baseline-orientation IMU signal, an orientation transformation matrix that transforms the current sensor signal from the current earbud orientation to the baseline earbud orientation; and
transform the current sensor signal from the current earbud orientation to the baseline earbud orientation using the orientation transformation matrix.

18. The media of claim 17, wherein estimating the orientation transformation matrix comprises estimating the orientation transformation matrix based on a grid search.

19. The media of claim 17, wherein estimating the orientation transformation matrix comprises estimating the orientation transformation matrix based on an iterative Markov chain Monte Carlo sampling until a stopping condition occurs.

20. The media of claim 17, wherein the instructions are further operable when executed by one or more processors to:
extract a plurality of features from (1) the transformed current sensor signal from the IMU of the earbud and from (2) an IMU sensor signal of a wrist-worn device; and
predict an activity of the user based on the extracted features.
